(19) United States
(12) Patent Application Publication
(10) Pub. No.: US 2025/0268471 A1
(43) Pub. Date: Aug. 28, 2025

(54) PREDICTIVE VISION BIOMETRICS

(71) Applicant: Alcon Inc., Fribourg (CH)

(72) Inventor: Paul R. Hallen, Colleyville, TX (US)

(21) Appl. No.: 19/019,727

(22) Filed: Jan. 14, 2025

Related U.S. Application Data
(60) Provisional application No. 63/556,496, filed on Feb. 22, 2024.

(51) Int. Cl. A61B 3/14; A61B 3/00

(52) U.S. Cl. CPC A61B 3/14; A61B 3/0025

(57) ABSTRACT

In certain embodiments, a system for predictive vision biometrics includes an ophthalmic multispectral imaging device, a memory including executable instructions, and a processor in communication with the memory and the ophthalmic multispectral imaging device. The ophthalmic multispectral imaging device is configured to generate ophthalmic multispectral data for a patient eye. The processor is configured to execute the instructions to determine a state of an eye condition in the patient eye based on the ophthalmic multispectral data, and to simulate one or more treatment scenarios for the patient eye based on the state of the eye condition and a model for the one or treatment scenarios.

### INTRODUCTION

Geographic atrophy (GA) is an advanced form of age-related macular degeneration that can result in the progressive and irreversible loss of retinal tissue. GA can lead to a loss of visual function over time. This often causes difficulty in performing daily tasks such as reading, recognizing faces, and driving, and ultimately has severe consequences on independence.

### SUMMARY

The present disclosure relates to diagnostic systems and methods, and more particularly, to systems and methods for predictive vision biometrics.

In certain embodiments, one general aspect includes a system for predictive vision biometrics. The system includes an ophthalmic multispectral imaging device, a memory including executable instructions, and a processor in communication with the memory and the ophthalmic multispectral imaging device. The ophthalmic multispectral imaging device is configured to generate ophthalmic multispectral data for a patient eye. The processor is configured to execute the instructions to determine a state of an eye condition in the patient eye based on the ophthalmic multispectral data, and to simulate one or more treatment scenarios for the patient eye based on the state of the eye condition and a model for the one or treatment scenarios.

In certain embodiments, another general aspect includes a method of operating an ophthalmic diagnostics system. The method includes generating, by an ophthalmic multispectral imaging device, ophthalmic multispectral data for a patient eye. The method also includes determining, by a processor in communication with the ophthalmic multispectral imaging device, a state of an eye condition in the patient eye based on the ophthalmic multispectral data. The method also includes simulating, by the processor, one or more treatment scenarios for the patient eye based on the state of the eye condition and a model for the one or treatment scenarios.

### BRIEF DESCRIPTION OF THE DRAWINGS

So that the manner in which the above-recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1 illustrates an example configuration of an ophthalmic diagnostics system, according to certain embodiments of the present disclosure.

FIG. 2 is a block diagram of various components of the ophthalmic diagnostics system of FIG. 1, according to certain embodiments of the present disclosure.

FIG. 3 illustrates an example of a process for using an ophthalmic diagnostics system for diagnosis and/or treatment of geographic atrophy, according to certain embodiments of the present disclosure.

FIG. 4A illustrates an example of an image that may be generated by a multispectral imaging device, according to certain embodiments of the present disclosure.

FIG. 4B illustrates an example of an image that may be generated by a simulator program, according to certain embodiments of the present disclosure.

FIG. 4C illustrates another example of an image that may be generated by a simulator program, according to certain embodiments of the present disclosure.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

For the purposes of promoting an understanding of the principles of the disclosure, reference will now be made to the implementations illustrated in the drawings, and specific language will be used to describe the same. It will nevertheless be understood that no limitation of the scope of the disclosure is intended. Any alterations and further modifications to the described systems, devices, instruments, methods, and any further application of the principles of the present disclosure are fully contemplated as would normally occur to one skilled in the art to which the disclosure relates In particular, the features, components, and/or steps described with respect to one implementation may be combined with the features, components, and/or steps described with respect to other implantations of the disclosure. For simplicity, in some instances the same reference numbers are used throughout the drawings to refer to the same or like parts.

With respect to treatment of GA, most current clinical trials aim to reduce the speed of lesion enlargement and corresponding vision loss using intravitreal injections. In other words, the objective is generally to slow the progression of GA as opposed to stopping or reversing it. In such a scenario, even a highly efficacious treatment may seem ineffective to the patient and, thus, not worth the cost, inconvenience, and discomfort.

The present disclosure describes examples of diagnostic systems and methods related to treatment of eye conditions such as GA. In certain embodiments, an ophthalmic diagnostics system can simulate multiple treatment scenarios for a patient eye based on a present state of an eye condition such as GA. The multiple treatment scenarios can include, for example, a scenario corresponding to a prescribed medication regimen and one or more other scenarios that deviate from the prescribed medication regimen. In various embodiments, information resultant from the simulations can be presented to the patient in an effort to encourage compliance with the prescribed medication regimen. Examples will be described relative to the Drawings.

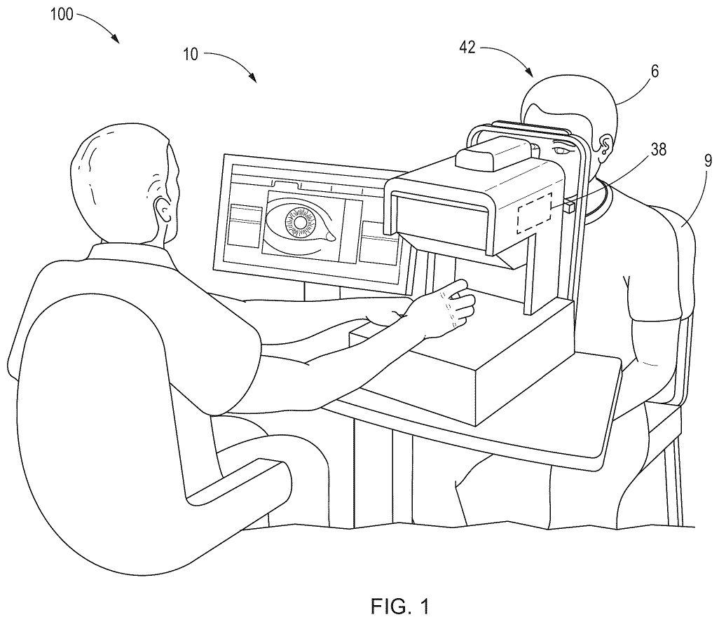

FIG. 1 illustrates an example configuration 100 of an ophthalmic diagnostics system 10 according to certain embodiments. The ophthalmic diagnostics system 10 may be used for different types of diagnostic and treatment procedures. For example, the ophthalmic diagnostics system 10 may be used for diagnosis or treatment of GA. In the configuration 100, the ophthalmic diagnostics system 10 is configured as a desktop imaging system in which the patient 42 sits in a chair 9. However, in certain other examples, the ophthalmic diagnostics system 10 may be configured for use in a setting where the patient is lying on a bed, or where the patient is standing.

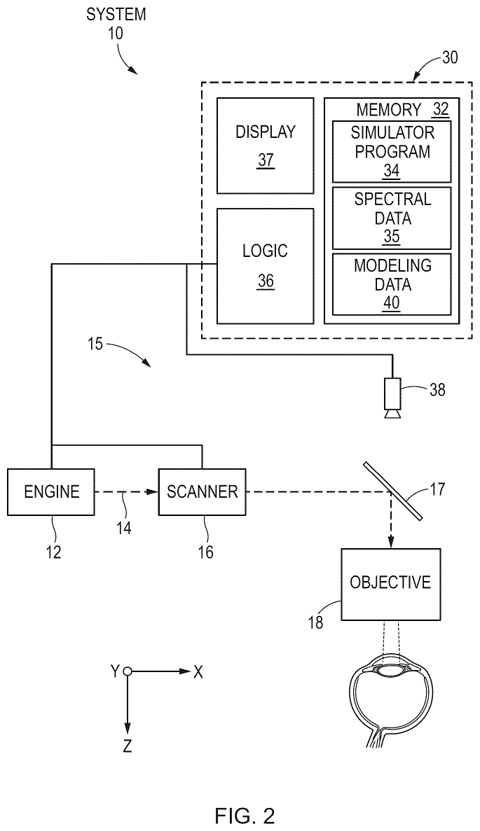

With reference now to FIG. 2, the ophthalmic diagnostics system 10 includes a multispectral imaging device 15 and a control computer 30, coupled as shown. The multispectral imaging device 15 includes controllable components, such as the one or more cameras 38, a multispectral imaging engine 12, a scanner 16, one or more optical elements 17, and/or a focusing objective 18, coupled as shown. The computer 30 includes logic 36, a memory 32, and a display 37, coupled as shown. The memory 32 can represent, for example, random access memory as well as storage. The storage can include, for example, a disk drive, a combination of fixed or removable storage devices (e.g., fixed disc drives), removable memory cards or optical storage, network attached storage (NAS), or a storage area-network (SAN). For ease of explanation, the following xyz-coordinate system is used: The z-direction is defined by the propagation direction of the imaging light, and the xy-plane is orthogonal to the propagation direction. Other suitable xyz-coordinate systems may be used.

With particular reference to the multispectral imaging device 15, the multispectral imaging engine 12 generates and emits imaging light, in multiple spectral bands, that is guided to tissue of an eye 120 of the patient 42. For example, the imaging light may be guided to a retinal surface 122 of the eye 120. In various embodiments, the multispectral imaging engine 12 can operate, for example, in blue, green, red, near-infrared, mid-infrared, far-infrared, thermal infrared and/or radar bands. In certain embodiments, the multispectral imaging engine 12 operates in monochromatic wavelengths that, in a typical embodiment, can reach a retinal pigment epithelium (RPE) layer. The scanner 16 laterally and/or longitudinally directs the imaging light. The lateral direction refers to directions orthogonal to the direction of beam propagation, i.e., the x, y directions. The scanner 16 may laterally direct the imaging light in any suitable manner. For example, the scanner 16 may include a pair of galvanometrically-actuated scanner mirrors that can be tilted about mutually perpendicular axes. As another example, the scanner 16 may include an electro-optical crystal that can electro-optically steer the imaging light.

The longitudinal direction refers to the direction parallel to the imaging light propagation, i.e., the z-direction. The scanner 16 may longitudinally direct the imaging light in any suitable manner. For example, the scanner 16 may include a longitudinally adjustable lens, a lens of variable refractive power, or a deformable mirror that can control the z-position of the beam focus. The components of the scanner 16 may be arranged in any suitable manner along a beam path, e.g., in the same or different modular units.

One or more optical elements 17 direct the imaging light towards the focusing objective 18. An optical element 17 can act on (e.g., transmit, reflect, refract, diffract, collimate, condition, shape, focus, modulate, and/or otherwise act on) the imaging light. Examples of optical elements include a lens, prism, mirror, diffractive optical element (DOE), holographic optical element (HOE), and spatial light modulator (SLM). In certain examples, the optical element 17 is a mirror or a dichroic mirror. The focusing objective 18 focuses the imaging light towards a portion of the eye 120, such as towards the retinal surface 122. In the example, focusing objective 18 is an objective lens, e.g., an f-theta objective.

Upon contact with the targeted eye tissues or structures (e.g., the retinal surface 122), the imaging light is converted to or results in the formation of a returned imaging light that may include one or a combination of reflection, scattering, fluorescence, auto fluorescence, or Raman spectra components. The multispectral imaging engine 12 receives the returned imaging light, from the eye 120, along the opposite direction of the imaging light. For example, the return light can include a portion of light that is reflected off the retinal surface 122 of the eye 120.

The multispectral imaging engine 12 can be configured to generate an image or images over any of the spectral bands discussed previously (e.g., monochromatic wavelengths), for example, to provide actionable feedback for storage, as described below in detail. For example, the multispectral imaging engine 12 can receive return light signals from the imaging light and, based on the return light, determine one or more spectral parameters of the targeted eye tissues/structures (e.g., the retinal surface 122), as well as other information, such as intensity and spectral information including, e.g., wavelength, polarization, and phase of the return light. In certain embodiments, the multispectral imaging engine can include a photodiode, avalanche photodiode, photomultiplier tube (PMT), or spectrometer.

The one or more cameras 38 can capture one or more images of the patient 42. In certain embodiments, the one or more cameras 38 include a hyperspectral camera that is able to provide a two-dimensional map of the retinal surface 122. In some embodiments, a multispectral camera (or other type of camera) may be used to provide a map of the retinal surface 122. In addition, or alternatively, the one or more cameras 38 may include any suitable device for generating a fundus image of the eye 120 and/or the retinal surface 122, and may include suitable magnification and focusing optics for performing that function. In addition, or alternatively, examples of the one or more cameras 38 include a video, interferometry, thermal imaging, ultrasound, and spectral imaging cameras. The one or more cameras 38 deliver image data, which represent recorded images of the eye 120 and/or the retinal surface 122. In some embodiments, the one or more cameras 38 can be an integral part of the multispectral imaging device 15. In other embodiments, the one or more cameras 38 can be separate from the multispectral imaging device 15.

The computer 30 controls components of the ophthalmic diagnostics system 10 in accordance with a simulator program 34. For example, the computer 30 controls components (e.g., the multispectral imaging engine 12, the scanner 16, the optical elements 17, and/or the focusing objective 18) to focus the imaging light of multispectral imaging engine 12 at a desired location on the eye 120, such as the retinal surface 122. In various embodiments, the simulator program 34 and its functionality, such as the focusing of the imaging light, can be directed by a user such as a medical professional.

The memory 32 stores programs and information used by the computer 30, and the computer 30 may access information from the memory 32. The memory 32 can include, for example, the simulator program 34, spectral data 35, and modeling data 40. The simulator program 34 can store, for example, computer-executable instructions for using the ophthalmic diagnostics system 10 for diagnosis and/or treatment of GA based on the spectral data 35 and the modeling data 40.

The spectral data 35 can include, for example, any data received or generated by the multispectral imaging device 15. The spectral data 35 can include, for example, an image or images based on the return light described previously, spectral parameters of targeted eye tissues/structures (e.g., the retinal surface 122), intensity information, spectral information such as wavelength, polarization and phase of the return light, combinations of the foregoing and/or the like. In addition, or alternatively, the spectral data 35 can include, for example, images (e.g., fundus images) or maps from the one or more cameras 38. Other examples of the spectral data 35 will be apparent to one skilled in the art after a detailed review of the present disclosure.

In some embodiments, the modeling data 40 can identify one or more treatment scenarios for an eye condition such as GA. The one or more treatment scenarios can each be defined by, for example, a medication regimen for a particular treatment (e.g., an injection using a named medication). For example, one treatment scenario may be defined by a prescribed medication regimen that involves taking a particular treatment at defined intervals (e.g., monthly, every other month, etc.) over a defined period of time (e.g., six months, one year, five years, etc.). Continuing this example, other treatment scenarios may correspond to deviations from the prescribed medication regimen (e.g., skipping one treatment over the period, skipping every other treatment, etc.). In still another example, a third treatment scenario may correspond to a deviation such that no treatments at all are taken over the period of time. In various examples, still other treatment scenarios can relate to different treatments, medication regimens, and/or periods of time. Other examples of treatment scenarios will be apparent to one skilled in the art after a detailed review of the present disclosure.

In some embodiments, the modeling data 40 can include data characterizing an effectiveness of the one or more treatment scenarios identified therein. For example, the modeling data 40 can include historical data related to an effectiveness of the one or more treatment scenarios in treating the eye condition in historical patients. For each treatment scenario, effectiveness can be characterized, at least in part, based on an initial state of the eye condition (i.e., before executing the treatment scenario) and a future/resultant state of the eye condition (i.e., after execution of the treatment scenario). For example, the initial and resultant states can each be expressed in terms of GA lesion size (e.g., measured surface area), visual acuity associated with the eye of the patient, combinations of the foregoing and/or the like. According to this example, the effectiveness of each treatment scenario can be expressed in terms of change in lesion size from the initial state to the future/resultant state, change in visual acuity from the initial state to the resultant state, combinations of the foregoing and/or the like. In various cases, the historical data can describe patients individually and/or aggregately in any suitable fashion. In some embodiments, the historical data can be, can include, and/or can be based on, data from clinical trials.

In some embodiments, the simulator program 34 can simulate treatment scenarios for a patient eye based on the spectral data 35 and the modeling data 40. The simulator program 34 can receive input data related to the patient eye, such as visual acuity and/or other information identifying or describing the patient, as well as cause the multispectral imaging device 15 to generate, for example, at least a portion of the spectral data 35. The simulator program 34 can determine a state of the eye condition (e.g., GA) in the patient eye based on the input data and the spectral data 35. In some embodiments, the simulation can include, for example, predicting a resultant state of the eye condition of the patient in response to hypothetical execution, by the patient, of the one or more treatment scenarios specified in the modeling data 40.

In some embodiments, the operation of the simulator program 34 can be at least partially rules-based according to the modeling data 40. For example, as part of simulating a treatment scenario specified in the modeling data 40, the simulator program 34 can apply a preconfigured adjustment to lesion size and/or visual acuity. For each simulated treatment scenario, the preconfigured adjustment that is applied can correspond to a predicted resultant state of the eye condition.

In addition, or alternatively, the operation of the simulator program 34 can be at least partially based in machine learning (e.g., supervised learning). For example, the modeling data 40 can include models trained on datasets for a large set of patients. According to this example, the datasets on which the modeling data 40 is trained can include records detailing sets of features such as treatment scenarios, initial states of an eye condition, and information describing each patient (e.g., age, gender, and ethnicity). Each record can further include, or be labeled with, a result state of any of the types described previously. Therefore, according to this example, the simulator program 34 can use the patient's state and/or available information describing the patient (e.g., age, gender, ethnicity, etc.) to predict or determine a resultant state for a given treatment scenario.

In some embodiments, the simulator program 34 can generate a simulated image based on its simulation. For example, the simulator program 34 can receive, from the one or more cameras 38, an input image (e.g., a fundus image) showing a lesion, having a measured lesion surface area, in the patient eye. According to this example, the simulation of one of the one or more treatment scenarios can yield a predicted resultant state that includes a predicted future lesion surface area, and the simulator program 34 can thereafter generate, from the input image, a simulated image that resizes (e.g., enlarges) a surface area of the lesion in correspondence to the predicted lesion surface area.

In some embodiments, the simulator program 34 can generate a comparative output based on its simulation functionality. In some cases, the comparative output can include information related to an initial state of the eye condition along with information related to one or more predicted resultant states for at least one of the one or more treatment scenarios. In addition, or alternatively, the comparative output can include information related to predicted resultant states for different treatment scenarios. For example, the simulator program 34 can predict resultant states for two or more treatment scenarios and can generate, as the comparative output, corresponding resultant information for each treatment scenario. The corresponding resultant information can include, for example, predicted resultant visual acuities, predicted lesion surface areas, simulated images of the type described above, and/or the like. For example, one treatment scenario may correspond to taking no treatments while another treatment scenario may correspond to following a prescribed medication regimen. Advantageously, in certain embodiments, even if it is predicted that the eye condition worsens under both treatment scenarios (e.g., lesion surface area increases and/or visual acuity worsens), the comparative output would illustrate that the worsening is less (e.g., much less) under the prescribed medication regimen, thereby encouraging the patient's compliance with the prescribed medication regimen. In various embodiments, similar comparative outputs may be generated for other treatment scenarios including, for example, various deviations from the prescribed medication regimen as described previously.

In some embodiments, the comparative output generated by the simulator program 34 can be backward-looking. In various embodiments, a backward-looking comparative output demonstrates what would have happened under a different treatment scenario than the one that occurred. For example, if the patient has executed the prescribed medication regimen, the comparative output can include a predicted resultant state of the eye condition of the patient if the patient had instead executed one or more different treatment scenarios that deviate from the prescribed medication regimen (e.g., skipping treatments or foregoing treatments entirely), thereby encouraging the patient's continued compliance. In another example, if the patient has executed a treatment regimen that deviates from the prescribed medication regimen (e.g., skipping treatments or foregoing treatments entirely), the comparative output can include a predicted resultant state of the eye condition of the patient if the patient had instead executed the prescribed medication regimen, thereby encouraging the patient's future compliance with the prescribed medication regimen.

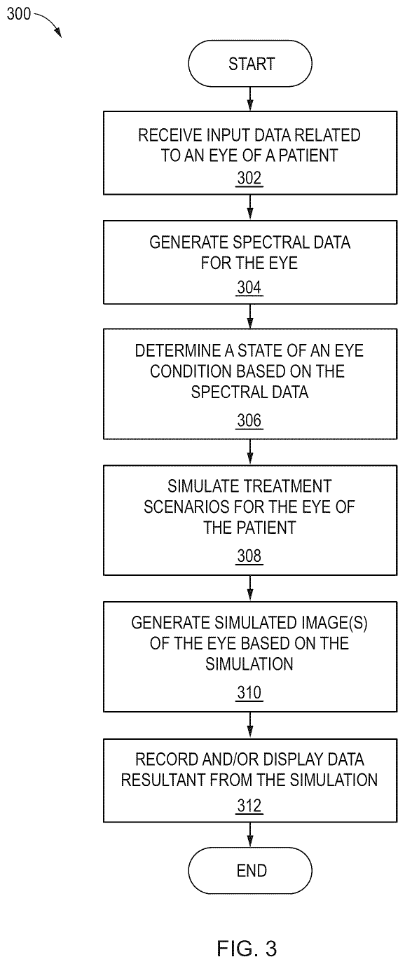

FIG. 3 illustrates an example of a process 300 for using an ophthalmic diagnostics system for diagnosis and/or treatment of GA. In certain embodiments, the process 300 can be implemented by any system that can process spectral imaging data. Although any number of systems, in whole or in part, can implement the process 300, to simplify discussion, the process 300 will be described in relation to example components of the ophthalmic diagnostics system 10 of FIG. 1.

At block 302, the computer 30 receives input data related to an eye of a patient. The input data can include, for example, a current visual acuity of the patient. In some cases, the current visual acuity can be received from an electronic medical record, obtained via data entry, and/or the like.

At block 304, the computer 30 generates, or causes generation of, spectral data (e.g., ophthalmic multispectral data) for the eye of the patient. In general, the spectral data can include any of the information or data described relative to FIGS. 1 and 2 that may be generated, for example, by the multispectral imaging device 15.

At block 306, the computer 30 determines a state of an eye condition in the patient eye based on the spectral data. For example, the computer 30 can measure, or cause to be measured, a surface area of a lesion in an image of the eye of the patient. At block 308, the computer 30 simulates one or more treatment scenarios (e.g., a first, second, third, etc. treatment scenario) for the eye of the patient based on the state of the eye condition and a model for the one or more treatment scenarios. For example, the one or more treatment scenarios may be simulated by the simulator program 34 as described relative to FIG. 2.

At block 310, the computer 30 generates treatment analysis data based on a result of the simulation at the block 308. The treatment analysis data can include, for example, one or more simulated images and/or one or more comparative outputs as described relative to FIG. 2. At block 312, the computer 30 records and/or displays data resultant from the simulation at the block 308. After block 312, the process 300 ends.

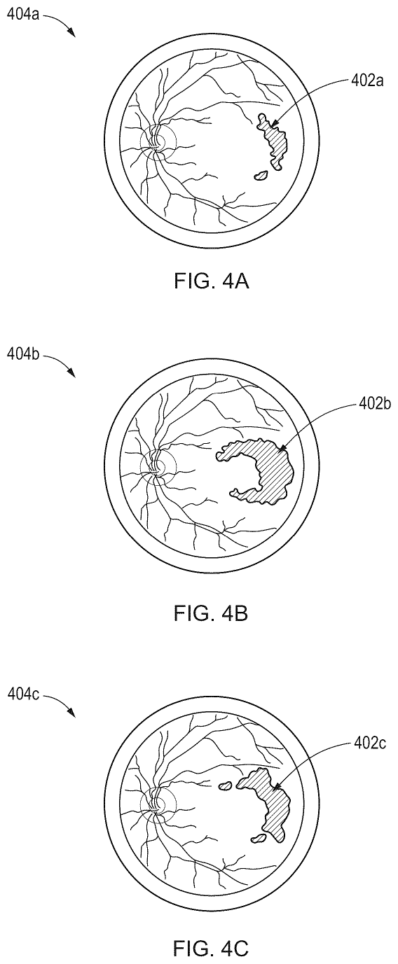

FIG. 4A illustrates an example of an image 400A that may be generated, for example, by the multispectral imaging device 15 of FIGS. 1B and 2. The image 400A illustrates a GA lesion 402A.

FIG. 4B illustrates an example of a simulated image 400B that may be generated, for example, by the simulator program 34 of FIG. 2. The simulated image 400B illustrates a GA lesion 402B.

FIG. 4C illustrates an example of a simulated image 400C that may be generated, for example, by the simulator program 34 of FIG. 2. The simulated image 400C illustrates a GA lesion 402C.

Referring to FIGS. 4A-C collectively, the images 400A, 400B and 400C may together form a comparative output that is generated, for example, as described relative to FIGS. 2 and 3. For example, the image 400A may correspond to an initial state of an eye condition in a patient eye while the images 400B and 400C each correspond to simulated images for different treatment scenarios. The image 400B, for example, may correspond to a predicted resultant state of following a prescribed medication regimen, while the image 400C may correspond to a predicted resultant state of following a treatment scenario that forgoes treatment. In various embodiments, the images 400B and 400C may be displayed to the patient in an effort to encourage the patient to follow the prescribed treatment regimen.

The above-disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

What is claimed is:

1. An ophthalmic system for predictive vision biometrics:
an ophthalmic multispectral imaging device configured to generate ophthalmic multispectral data for a patient eye;
a memory comprising executable instructions; and
a processor in communication with the memory and the ophthalmic multispectral imaging device and configured to execute the executable instructions to:
determine a state of an eye condition in a patient eye based on the ophthalmic multispectral data; and
simulate one or more treatment scenarios for the patient eye based on the state of the eye condition and a model for the one or more treatment scenarios.

2. The ophthalmic system of claim 1, wherein the processor is further configured to execute the instructions to at least one of record or display data resultant from the simulated one or more treatment scenarios.

3. The ophthalmic system of claim 1, wherein the simulation comprises a prediction of a resultant state of the eye condition in response to hypothetical execution of the one or more treatment scenarios.

4. The ophthalmic system of claim 1, wherein:
the simulation comprises:
a prediction of a first resultant state of the eye condition in response to hypothetical execution of a first treatment scenario; and
a prediction of a second resultant state of the eye condition in response to hypothetical execution of a second treatment scenario; and
the processor is further configured to execute the instructions to generate a comparative output with respect to the first resultant state and the second resultant state.

5. The ophthalmic system of claim 4, wherein:
the first treatment scenario corresponds to a prescribed medication regimen; and
the second treatment scenario corresponds to a deviation from the prescribed medication regimen.

6. The ophthalmic system of claim 1, wherein the model is based on historical data related to an effectiveness of the one or more treatment scenarios in treating the eye condition in other patients.

7. The ophthalmic system of claim 1, wherein the ophthalmic multispectral imaging device operates over a monochromatic wavelength.

8. The ophthalmic system of claim 1, wherein the processor is further configured to execute the instructions to generate a simulated image of the patient eye based on a result of the simulation.

9. The ophthalmic system of claim 1, wherein:
the determination of a state comprises measuring a surface area of a lesion in an image of the patient eye; and
the simulation comprises predicting a future surface area of the lesion in response to hypothetical execution of the one or more treatment scenarios.

10. The ophthalmic system of claim 1, further comprising receiving input data comprising a visual acuity associated with the patient eye, wherein the simulating comprises predicting a future visual acuity in relation to the patient eye in response to hypothetical execution of the one or more treatment scenarios.

11. A method for predictive vision biometrics, the method comprising:
generating, by an ophthalmic multispectral imaging device, ophthalmic multispectral data for a patient eye;
determining, by a processor in communication with the ophthalmic multispectral imaging device, a state of an eye condition in the patient eye based on the ophthalmic multispectral data; and
simulating, by the processor, one or more treatment scenarios for the patient eye based on the state of the eye condition and a model for the one or treatment scenarios.

12. The method of claim 11, further comprising at least one of recording or displaying, by the processor, data resultant from the simulated one or more treatment scenarios.

13. The method of claim 11, wherein the simulating comprises predicting a resultant state of the eye condition in response to hypothetical execution of the one or more treatment scenarios.

14. The method of claim 11, wherein:
the simulating comprises:
predicting a first resultant state of the eye condition in response to hypothetical execution of a first treatment scenario; and
predicting a second resultant state of the eye condition in response to hypothetical execution of a second treatment scenario; and
the method further comprises generating, by the processor, a comparative output with respect to the first resultant state and the second resultant state.

15. The method of claim 11, wherein:
the determining a state comprises measuring a surface area of a lesion in an image of the patient eye; and
the simulating comprises predicting a future surface area of the lesion in response to hypothetical execution of the one or more treatment scenarios.
